(19) United States
(12) Patent Application Publication
(10) Pub. No.: US 2007/0172519 A1
(43) Pub. Date: Jul. 26, 2007

(54) Instant Sterile Adhering Functional Patch Manufacturing Machine

(76) Inventor: Chung-King Hsu, Taipei (TW)

Correspondence Address: WPAT, PC;INTELLECTUAL PROPERTY ATTORNEYS, 2030 MAIN STREET, SUITE 1300, IRVINE, CA, 92614, US

(21) Appl. No.: 11/623,264

(22) Filed: Jan. 15, 2007

(30) Foreign Application Priority Data
Jan. 24, 2006 (TW) 095102636

(51) Int. Cl. A61F 13/02; G05G 15/00; A61K 36/889

(52) U.S. Cl. 424/448; 424/725.1; 156/350

(57) ABSTRACT

An instant sterile adhering functional patch manufacturing machine is disclosed. This machine can be installed at any location. After a consumer deposits the coins and presses the start button, the process is then initiated at the machine. Using the internal operating mechanisms contained inside the main body, the extract solution, which has a specific formulation, is dosed onto an adhering patch under a sterile environment. And later, after the appropriate packaging and printing of the associated text or patterns, the finished packaged and printed product is delivered out of the main body to allow the consumer to instantly receive a fresh adhering functional patch.

### BACKGROUND OF THE INVENTION

1. Field of the Invention

The present invention relates to an adhering functional patch manufacturing and vending machine able to manufacture instantly on the spot.

2. The Prior Arts

The conventional so-called adhering functional patch pertains to a patch which incorporates a unique formulation to thereby form the adhering stick-on patch. The adhering patch can be made to adhere to a person's skin surface, for allowing the provided formulation to be seeped into the human body to produce an expected effect. Examples of such adhering patch are facial mask, chest mask, eye mask, pain relief patch, etc. Using facial mask as an example, the conventional facial mask are all pre-made and pre-packaged at factories, and then sold through distribution channels arranged throughout the market. Therefore, not only is the product cost to be excessive, but additionally, the brand name can be easily pirated. Furthermore, typical facial masks are mostly found in the form of having several pieces packaged inside a single box, in which the consumer, after purchasing, would not be able to use in a short period of time. Although the product would not have a problem of becoming defective within the stated shelf life, but nevertheless, the contained ingredients would no longer be as fresh as compared to when it was first made, especially as it is approaching expiration date, the consumer then becomes more and more uneasy about the product.

### SUMMARY OF THE INVENTION

A main objective of the present invention is to resolve the higher manufacturing cost of the aforementioned conventional adhering functional patch.

Another objective of the present invention is to resolve an issue related to the aforementioned conventional adhering functional patch product distribution method, in which its raw material and brand can easily be pirated, thereby bringing about undesirable effects during product consumption.

Yet another objective of the present invention is to resolve another issue related to the aforementioned conventional adhering functional patch product distribution method, in which the consumer is unable to obtain the freshest product.

A main feature of the present invention is to provide a type of adhering functional patch manufacturing machine having instant manufacturing and vending capabilities after accepting a consumer's product selection. This machine can be installed at any location. After the consumer has inserted coins and pressed the buttons to initiate operation, various devices/units inside the main body, operating under a plurality of mechanisms under a sterile environment, would take the extract solution, which contains the formulation, to join to the adhering patch. In addition, after performing appropriate packaging and printing of the associated text or patterns, the finished product is to be brought out of the machine to allow the consumer to then instantly receive a fresh adhering functional patch.

An objective of the present invention is to incorporate an adhering patch storage container, an extract solution storage container, a packaging material storage container, a sealing device, a printing device, and a plurality of conveying devices inside a main body. The adhering patch storage container is used for dosing/dispensing the pre-cut adhering patch, which has water absorption; the extract solution storage container is for holding the pre-made liquid extract solution (which is the extract solution) containing a specific formulation. The extract solution storage container includes a liquid outlet. The liquid outlet is set up with a control valve used for controlling the flow output of the extract solution inside the extract solution storage container. Each of the aforementioned devices and mechanisms is configured with a pre-established computer program and electrical circuit. After the consumer inserted the coins and has followed along the operating procedure, using the aforementioned computer program and electrical circuit for controlling one of the conveying devices, an adhering patch is conveyed close to the liquid outlet of the extract solution storage container. In addition, using the control valve of the liquid outlet, a sufficient dosage of the extract solution is appropriately provided onto the adhering patch by means of the capillary effect for dispensing/dosing the extract solution dispersing onto the entire adhering patch. Another set of conveying device, at the same time, is used to convey the packaging material to the sealing device, and to convey the adhering patch containing the extract solution from another conveying device onto the packaging material of the sealing device, and to seal the adhering patch using the sealing device, and then using yet another conveying device for conveying to the printing device. Then at the packaging material surface, the required text (such as for example, date of manufacture, expiration date, etc) or patterns are printed and finally conveyed to the vending opening at the lower portion of the main body for pick up by the consumer.

Another objective of the present invention is to set up a sterile area inside the main body for allowing the aforementioned adhering patch to perform the process of dosing of the extract solution and all the way to the packaging process to be fully completed inside the sterile area for preventing contamination of the adhering patch.

An objective of the present invention pertaining to the aforementioned sterile area is to be able to adopt several options. For example, the spraying method for spraying the bactericidal/bacteriostatic solution inside an enclosed space can be adopted for eliminating or controlling the bacterial growth. And additionally, ultraviolet radiation or ozone producing method can be adopted to arrive at the same objective.

Yet another objective of the present invention is to be able to set up a process display panel equipment with multimedia capability to be disposed on the cover surface of the machine for allowing the consumer to clearly understand the manufacturing processes and steps after inserting the coins to operate the machine, thus eliminating tendency for boredom due to lack of understanding. Furthermore, the cover or the main body surface can also be set up with advertisement printings, or with the use of an LCD panel having multimedia effects for providing advertisement media.

### BRIEF DESCRIPTION OF THE DRAWINGS

The present invention will be apparent to those skilled in the art by reading the following detailed description of embodiments thereof, with reference to the attached drawings, in which:

FIG. 1 is a perspective view showing an adhering functional patch manufacturing machine in accordance with the present invention;

FIG. 2 is a block diagram showing an adhering functional patch manufacturing machine according to an embodiment of the present invention;

FIG. 3 is a schematic view showing the dosing of the extract solution onto the adhering functional patch according to the embodiment of the present invention; and

FIG. 4 is a perspective view showing the packaged finished product of the extract solution containing adhering functional patch using a packaging material.

### DETAILED DESCRIPTION OF THE EMBODIMENTS

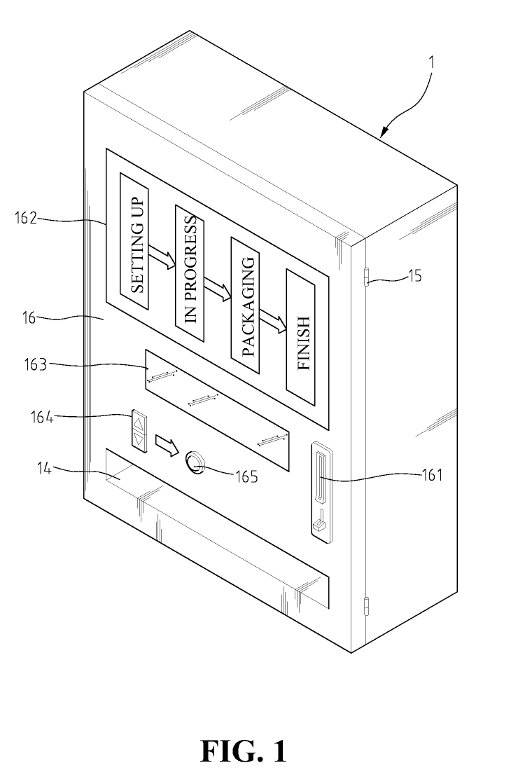

Referring to FIG. 1, an adhering functional patch manufacturing machine according to the present invention can be installed in any location, and having the capability to manufacture and to provide vending at the field/site. The front of a main body 1 of the machine is coupled to a cover 16 using a hinge 15, thus allowing the cover 16 to be able to correspondingly rotate with respect to the main body 1 when opening or closing the main body 1. When the cover 16 is opened, various items can be replenished, or maintenance can be performed. A coin deposit slot 161, a process display panel 162, a display 163, a quantity control button 164, a start button 165, and a vending opening 14 can be disposed on the aforementioned cover 16. After the consumer deposits the coins into the coin deposit slot 161, the display 163 is to display the pending purchase quantity request information. At this time, the consumer is to operate the quantity control button 164 to determine the purchase amount, and then to press the start button 165 for performing the manufacturing and packaging procedures, in which the process status can be indicated with lights from the aforementioned process display panel 162. The finished product, after manufacturing and packaging, would then drop to the vending opening 14 for pick up by the consumer.

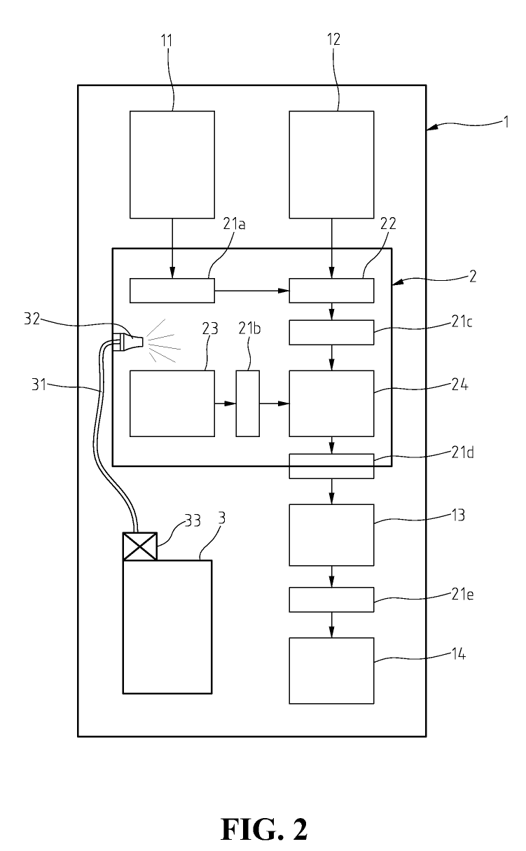

Referring further to FIG. 2, according to a preferred embodiment of the present invention, an adhering patch storage container 11, an extract solution storage container 12, an extract solution dosage area 22, a packaging material storage container 23, a sealing device 24, a printing device 13, a vending opening 14 that is connected to the outside of the cover 16, and a first through fifth conveying device 21a-21e are contained inside the main body 1. In addition, a sterile area 2 is provided for containing the first to the third conveying device 21a-21c, the extract solution dosage area 22, the packaging material storage container 23, and the sealing device 24.

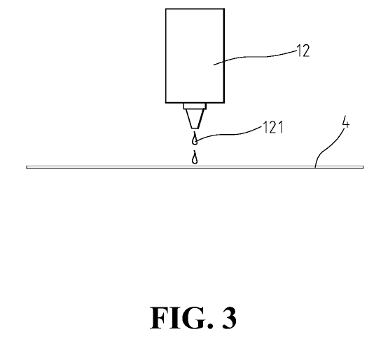

The aforementioned adhering patch storage container 11 is for holding the pre-fabricated adhering patch (such as the facial mask), and to be placed in a stacked manner. The lower portion of the aforementioned extract solution storage container 12 includes a liquid outlet, in which the outlet nozzle for the liquid outlet is set up with a control valve for regulating the flowrate of the extract solution inside the extract solution storage container 12 (not illustrated in the figures). The aforementioned first through fifth conveying device 21a-21e can be of a mechanical arm or a conveyor belt. The present invention is using the first conveying device 21a to convey an adhering patch to the extract solution dosage area 22, and using the control valve of the extract solution storage container 12 to regulate the opening of the liquid outlet to transport a sufficient amount of the extract solution 121 (referring to FIG. 3) for allowing the extract solution 121 to be dispensed/dosed in the center portion of the adhering patch 4, and using capillary principle for spreading the extract solution to the entire adhering patch 4. The aforementioned adhering patch 4 can be made from non-woven fabric or a paper material.

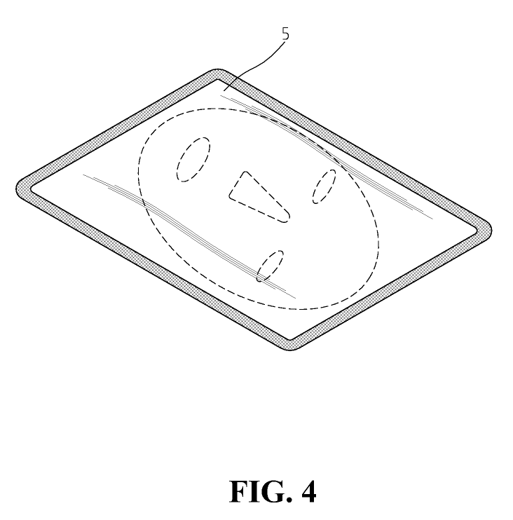

The aforementioned packaging material storage container 23 is used for providing the packaging material for use during the sealing of the adhering patch 4. The sealing device 24 is used at a later processing procedure after the steps performed in the extract solution dosage area 22. As the adhering patch 4 is added with the extract solution, the second conveying device 21b is to convey a packaging material to the sealing device 24 and to wait. After the completion of adding extract solution onto the adhering patch 4, the third conveying device 21c then conveys the adhering patch 4 to the packaging material of the sealing device 24, and using the sealing device 24 to package and seal the adhering patch inside the packaging material 5 (as illustrated in FIG. 4). The packaged finished product is conveyed using the fourth conveying device 21d to the printing device 13. The printing device 13 then prints the product information (such as, for example, date of manufacture, expiration date) onto the packaging material, and then using the fifth conveying device 21e to transport the printed finished product to the vending opening 14, where the consumer can collect the finished product from the vending opening 14.

Referring to the sterile area 2 in accordance with the preferred embodiment of the present invention, a bactericidal/bacteriostatic device is to be disposed inside the sterile area 2. The bactericidal/bacteriostatic device can take on several optional forms, in which one possible option can be the placing of a spray nozzle 32 inside the sterile area 2, and the disposing of a storage container 3 outside the sterile area 2 for holding the bactericidal/bacteriostatic solution. The storage container 3 and the spray nozzle 32 are coupled using a transfer tubing 31, and the transfer tubing 31 is disposed with a pump 33 for pumping out the bactericidal/bacteriostatic solution from inside the storage container 3 and for spraying out from the spray nozzle 32. To control by using the computer program, the pump 33 is allowed to timely operate and to provide sufficient amount of bactericidal/bacteriostatic solution to be sprayed inside the sterile area 2 to arrive at the sterilization or bacterial growth inhibition purposes. The aforementioned bactericidal/bacteriostatic solution can be selected from natural bamboo vinegar, TiO₂ catalyst, bleach, and alcohol, etc.

The present invention can also use a ultraviolet radiation producing device or an ozonator (not shown in the figures) located inside the sterile area 2 for achieving the same level of bactericidal or bacteriostatic effects.

Although the present invention has been described with reference to the embodiments thereof, it is apparent to those skilled in the art that a variety of modifications and changes may be made without departing from the scope of the present invention which is intended to be defined by the appended claims.

What is claimed is:

1. An instant sterile adhering functional patch manufacturing machine, comprising:
an adhering patch storage container, for holding a plurality of adhering patches in sheet form;
an extract solution storage container, comprising of a liquid outlet, wherein the liquid outlet is disposed with a control valve for regulating the flow output of the extract solution from inside the extract solution storage container;
a first conveying device, for conveying the adhering patch inside the adhering patch storage container to a position close to the liquid outlet of the extract solution storage container for allowing the adhering patch to receive the extract solution from the liquid outlet;
a packaging material storage container, for holding the packaging material;
a sealing device;
a second conveying device, for conveying the packaging material to the sealing device;
a third conveying device, for conveying the adhering patch containing the extract solution to the packaging material of the sealing device, thereby sealing the adhering patch inside the packaging material using the sealing device;
a sterile area, containing the first conveying device, the second conveying device, the third conveying device, the packaging material storage container, and the sealing device; and the sterile area is disposed with a bactericidal/bacteriostatic device;
a printing device;
a fourth conveying device, for conveying the adhering patch, already sealed using the packaging material, to the printing device;
a vending opening, for holding the packaged finished product;
a fifth conveying device, for conveying the printed finished product to the vending opening;
a main body, for holding all of the aforementioned elements and devices, disposed with a process display panel and coin deposit slot, and is configured to a computer program and circuit design to control the operation of the machine.

2. The instant sterile adhering functional patch manufacturing machine as claimed in claim 1, wherein, the adhering patch is made of a nonwoven fabric.

3. The instant sterile adhering functional patch manufacturing machine as claimed in claim 1, wherein the adhering patch is of a sheet form made of paper.

4. The instant sterile adhering functional patch manufacturing machine as claimed in claim 2, wherein, the adhering patch is a facial mask conforming to a human facial contour.

5. The instant sterile adhering functional patch manufacturing machine as claimed in claim 1, wherein the first conveying device, the second conveying device, the third conveying device, the fourth conveying device, and the fifth conveying device are of a plurality of mechanical arms.

6. The instant sterile adhering functional patch manufacturing machine as claimed in claim 1, wherein the first conveying device, the second conveying device, the third conveying device, the fourth conveying device, and the fifth conveying device are of a plurality of conveyor belts.

7. The instant sterile adhering functional patch manufacturing machine as claimed in claim 1, wherein the bactericidal/bacteriostatic device containing a spray nozzle disposed inside the sterile area; and the storage container for holding the bactericidal/bacteriostatic solution disposed outside of the sterile area; the storage container and the spray nozzle are coupled using a transfer tubing; the transfer tubing is disposed with a pump for dispensing the bactericidallbacteriostatic solution from inside the storage container and for spraying out from the spray nozzle.

8. The instant sterile adhering functional patch manufacturing machine as claimed in claim 1, wherein the bactericidal/bacteriostatic device is an ultraviolet radiation producing device.

9. The instant sterile adhering functional patch manufacturing machine as claimed in claim 1, wherein the bactericidal/bacteriostatic device is an ozonator.

10. The instant sterile adhering functional patch manufacturing machine as claimed in claim 7, wherein the bactericidal/bacteriostatic solution is a natural bamboo vinegar.

11. The instant sterile adhering functional patch manufacturing machine as claimed in claim 7, wherein the bactericidal/bacteriostatic solution containing a TiO₂ catalyst.

12. The instant sterile adhering functional patch manufacturing machine as claimed in claim 7, wherein the bactericidal/bacteriostatic solution is a bleach.

13. The instant sterile adhering functional patch manufacturing machine as claimed in claim 7, wherein the bactericidal/bacteriostatic solution is an alcohol.

14. The instant sterile adhering functional patch manufacturing machine as claimed in claim 1, wherein the main body is joined with a cover, and the surface of the cover is disposed with a display, and a process display panel for displaying the operating status.
